(19) United States
(12) Patent Application Publication
(10) Pub. No.: US 2023/0313228 A1
(43) Pub. Date: Oct. 5, 2023

(54) CELL LINE FOR USE IN PRODUCING RECOMBINANT ADENOVIRUSES

(71) Applicant: Oxford Genetics Limited, Oxford Oxfordshire (GB)

(72) Inventor: Ryan Cawood, Oxford Oxfordshire (GB)

(21) Appl. No.: 18/021,684

(22) PCT Filed: Aug. 20, 2021
(86) PCT No.: PCT/GB2021/052161

§ 371 Date: Feb. 16, 2023

(30) Foreign Application Priority Data
Aug. 21, 2020 (GB) 2013060.5

(51) Int. Cl. C12N 15/86; C12N 15/113

(52) U.S. Cl. CPC C12N 15/86; C12N 15/1131; C12N 2710/10351; C12N 2710/10343; C12N 2310/11; C12N 2750/14122

(57) ABSTRACT

The present invention relates to a cell for use in producing recombinant adenoviruses (AVs), wherein DNA molecules which are capable of producing antisense RNAs against AAV cap and AAV rep mRNAs are stably integrated into the cell's genome. The invention also relates to processes for the production of such cells and processes for using such cells in the production of recombinant AVs.

### CROSS-REFERENCE

This application is a section 371 U.S. national phase of PCT/GB2021/052161, filed Aug. 20, 2021, which claims priority from GB 2013060.5, filed Aug. 21, 2020, both which are incorporated by reference in their entirety.

### FIELD OF THE INVENTION

The present invention relates to a cell for use in producing recombinant adenoviruses (AVs), wherein DNA molecules which are capable of producing antisense RNAs against AAV cap and AAV rep mRNAs are stably integrated into the cell's genome. The invention also relates to processes for the production of such cells and processes for using such cells in the production of recombinant AVs.

### STATEMENT REGARDING SEQUENCE LISTING

A computer readable form of the Sequence Listing is filed with this application by electronic submission and is incorporated into this application by reference in its entirety. The sequence listing submitted herewith is contained in the text file created Feb. 16, 2023, entitled “23-0200-WO-US_Sequence-Listing_ST25.txt” and 112 kilobytes in size.

### BACKGROUND OF THE INVENTION

Adeno-associated viruses (AAVs) are single-stranded DNA viruses that belong to the Parvoviridae family. It is a non-pathogenic virus that generates only a limited immune response in most patients. The virus cellular and tissue tropism is defined by the capsid on the surface of the AAV particles. Some capsids allow infection of a broad range of host cells, including both dividing and non-dividing cells, whilst others are considerably more restricted. Some do not infect standard production or manufacturing cell lines such as HEK293 cells.

Over the last few years, vectors derived from AAVs have emerged as an extremely useful and promising mode of gene delivery. This is owing to the following properties of these vectors:
AAVs are small, non-enveloped viruses and they have only two native genes (rep and cap). Thus, they can be easily manipulated to develop vectors for different gene therapies. This is achieved by the removal of the rep and cap genes in the AAV genome and replacing these sequences with exogenous sequences (transgenes) that may provide therapeutic benefit to a patient.
AAV particles are not easily degraded by shear forces, enzymes or solvents. This facilitates easy purification and final formulation of these viral vectors.
AAVs are non-pathogenic and have a low immunogenicity. The use of these vectors further reduces the risk of adverse inflammatory reactions. Unlike other viral vectors, such as lentivirus, herpes virus and adenovirus, AAVs are harmless and are not thought to be responsible for causing any human disease.
Genetic sequences up to approximately 4500 bp can be delivered into a patient using AAV vectors.
Whilst wild-type AAV vectors have been shown to sometimes insert genetic material into human chromosome 19, this property is generally eliminated from most AAV gene therapy vectors by removing rep and cap genes from the viral genome. In such cases, the virus remains in an episomal form within the host cells. These episomes remain intact in non-dividing cells, while in dividing cells they are lost during cell division.

The native AAV genome comprises two genes each encoding multiple open reading frames (ORFs): the rep gene encodes non-structural proteins that are required for the AAV life-cycle and site-specific integration of the viral genome; and the cap gene encodes the structural capsid proteins.

In addition, these two genes are flanked by inverted terminal repeat (ITR) sequences consisting of 145 bases that have the ability to form hairpin structures. These hairpin sequences are required for the primase-independent synthesis of a second DNA strand and the integration of the viral DNA into the host cell genome.

In order to eliminate any integrative capacity of the virus, some recombinant AAV vectors remove rep and cap from the DNA of the viral genome. To produce such vectors, the desired transgene(s), together with a promoter(s) to drive transcription of the transgene(s), is inserted between the inverted terminal repeats (ITRs); and the rep and cap genes are provided in trans from either a second plasmid or a helper virus encoding the rep and or cap genes. Helper genes such as adenovirus E4, E2a and VA genes are also provided by either a plasmid or a helper virus. rep, cap and helper genes may be provided on additional plasmids that are transfected into cells or via a helper virus.

Traditionally, the production of AAV vectors has been achieved through a number of different routes.

Initially, AAV was generated using wild-type (WT) Adenovirus serotype 5 whilst transfecting cells with plasmids encoding the rep and cap genes and the AAV genome. This allowed the WT adenovirus to provide a number of factors in trans that facilitated AAV virus replication. However, there are a number of limitations to this approach: for example, each batch of AAV must be separated from the Adenoviral (AV) particles after manufacture to provide a pure product and ensuring that all Ad5 has been removed is challenging. Moreover, the fact that during production the cell is devoting considerable resources to the production of Adenoviral particles rather than AAV is also undesirable.

In other systems, stable packing cells lines expressing the rep and cap genes have been used. In such systems, the rep and cap genes are integrated into the cell genomes, hence obviating the need for plasmid-based rep and cap genes. However, these genes are usually only integrated at low frequency (e.g. 1-2 copies per cell) due to their inherent toxicity. These systems require the infection with adenoviral vectors.

### SUMMARY OF THE INVENTION

The present invention relates to a cell for use in producing recombinant adenoviruses (AVs), wherein DNA molecules which are capable of producing antisense RNAs against AAV cap and AAV rep mRNAs are stably integrated into the cell's genome. The invention also relates to processes for the production of such cells and processes for using such cells in the production of recombinant AVs.

More recently, the adenovirus-based systems have been replaced with plasmids encoding the sections of the Adenovirus genome required for AAV production.

The inventors have for the first managed to insert both cap and rep genes into an adenoviral vector. These vectors are stable and can be used for AAV manufacture. However, it was noted that some variants of Cap and Rep polypeptides can reduce the growth of the adenovirus in comparison to an adenovirus that does not contain the rep and/or cap genes for some variants of Rep and Cap. It was also noted that high level of expression of both genes can significantly impact adenovirus replication. This could be due to cytotoxic effects of AAV Rep and/or Cap polypeptides in the cells or that the expression levels of the components are too high. It has also been noted that cytotoxicity from some Cap variants can be higher than others and this may pose a problem for recombinantly-engineered Cap variants when encoded within an adenoviral vector.

The inventors have recognised, therefore, that a system in which the expression of AAV Rep and Cap polypeptides could be regulated, i.e. switched on and off, would be advantageous when producing adenoviral vectors encoding either of them. This would allow for the growth of adenoviral vectors encoding the AAV Rep and Cap polypeptides in cells without any detriment to the adenovirus yield. Then, when AAVs are to be produced, this can be achieved in a cell line that does not contain the system that represses production of the AAV Rep and/or Cap polypeptides.

The inventors have now developed a cell for use in producing recombinant adenoviruses which encode AAV Rep and/or AAV Cap polypeptides, wherein the cell encodes antisense RNA molecules against the mRNAs encoding the Rep and/or Cap polypeptides.

It is an object of the invention therefore to provide a process for producing recombinant adenoviruses in a host cell, wherein the host cell is capable of regulating the production of AAV Cap and Rep polypeptides in the host cell.

It is also an object of the invention to provide a cell for the production of recombinant AV particles and a process for producing such a cell.

### DESCRIPTION OF THE DRAWINGS

FIG. 1 represents the growth of adenoviral vectors containing mRNAs encoding AAV rep and cap genes (“TERA-Cap2-Rep”) or without (“Ad5-E1/E3 deleted”), in the presence of siRNAs against various AAV rep (“Rep-E”,“Rep-F”) and cap (“Cap2-A”, etc.) mRNAs. “Scrambled” represents a sequence-scrambled siRNA, as a control that does not bind Rep or Cap mRNAs.

### DETAILED DESCRIPTION OF THE INVENTION

In one embodiment, the invention provides a cell which comprises:
(a) a first DNA molecule which comprises:
   (i) a first promoter, operably-associated with
   (ii) a DNA molecule which encodes an antisense RNA which is capable of binding to an AAV Cap mRNA,
and/or
(b) a second DNA molecule which comprises:
   (i) a second promoter, operably-associated with
   (ii) a DNA molecule which encodes an antisense RNA which is capable of binding to an AAV Rep mRNA,
and wherein the first and/or second DNA molecules are stably integrated into the cell's genome.

### Details of Sequences

The following sequences are given in the Sequence Listing, which forms part of the description of this patent application.

| SEQ ID NO: | Description | Form |
|---|---|---|
| 1 | AAV1 capsid | Nucleotide |
| 2 | AAV1 capsid | Amino acid |
| 3 | AAV2 capsid | Nucleotide |
| 4 | AAV2 capsid | Amino acid |
| 5 | AAV3 capsid | Nucleotide |
| 6 | AAV3 capsid | Amino acid |
| 7 | AAV4 capsid | Nucleotide |
| 8 | AAV4 capsid | Amino acid |
| 9 | AAV5 capsid | Nucleotide |
| 10 | AAV5 capsid | Amino acid |
| 11 | AAV6 capsid | Nucleotide |
| 12 | AAV6 capsid | Amino acid |
| 13 | AAV7 capsid | Nucleotide |
| 14 | AAV7 capsid | Amino acid |
| 15 | AAV8 capsid | Nucleotide |
| 16 | AAV8 capsid | Amino acid |
| 17 | AAV9 capsid | Nucleotide |
| 18 | AAV9 capsid | Amino acid |
| 19 | AAV2 rep gene | Nucleotide |
| 20 | AAV2 Rep78 | Nucleotide |
| 21 | AAV2 Rep78 | Amino acid |
| 22 | AAV2 Rep68 | Nucleotide |
| 23 | AAV2 Rep68 | Amino acid |
| 24 | AAV2 Rep52 | Nucleotide |
| 25 | AAV2 Rep52 | Amino acid |
| 26 | AAV2 Rep40 | Nucleotide |
| 27 | AAV2 Rep40 | Amino acid |
| 28 | TetR binding site | Nucleotide |
| 29 | Modified AV Major | Nucleotide |
| | Late Promoter | |
| 30 | Modified AV Major | Nucleotide |
| | Late Promoter | |
| 31 | siRNA | RNA |
| 32 | siRNA | RNA |
| 33 | siRNA | RNA |
| 34 | siRNA | RNA |
| 35 | siRNA | RNA |
| 36 | siRNA | RNA |
| 37 | siRNA | RNA |
| 38 | siRNA | RNA |
| 39 | siRNA | RNA |
| 40 | siRNA | RNA |
| 41 | siRNA | RNA |
| 42 | siRNA | RNA |

The cell may be a recombinant cell. The cell may be an isolated cell. The cell may be a cell from a cell line. The invention includes cell lines consisting of or comprising the cells of the invention.

The cells may be primary or immortalised cells. The cell is preferably a mammalian cell. Examples of mammalian cells include those from any organ or tissue from humans, mice, rats, hamsters, monkeys, rabbits, donkeys, horses, sheep, cows and apes. Preferably, the cell is a human cell.

Preferably, the cell is from an adenovirus production cell line or an adenovirus manufacturing cell line, i.e. a cell line that is either highly infectable by adenovirus, or which contains integrated copies of the adenovirus E1A and E1B genes.

Preferred first host cells include HEK-293, HEK 293T, HEK-293E, HEK-293 FT, HEK-293S, HEK-293SG, HEK-293 FTM, HEK-293SGGD, HEK-293A MDCK, C127, A549, HeLa, CHO, mouse myeloma, PerC6, 911 and Vero cell lines, and derivatives thereof. HEK-293 cells have been modified to contain the E1A and E1B genes; this obviates the need for the corresponding proteins to be supplied on a Helper Plasmid. PerC6 and 911 cells contain a similar modification and can also be used. Most preferably, the cell is a HEK293, HEK293T, HEK293A, PerC6 or 911 cell.

AAV Cap mRNA is produced from an AAV cap gene. As used herein, the term “cap gene” refers to a gene that encodes one or more open reading frames (ORFs), wherein each of said ORFs encodes an AAV Cap structural protein, or variant or derivative thereof. These AAV Cap structural proteins (or variants or derivatives thereof) form the AAV capsid. The three Cap proteins are VP1, VP2 and VP3, which are generally 87 kDa, 72 kDa and 62 kDa in size, respectively. Hence the cap gene is one which encodes the three Cap proteins VP1, VP2 and VP3. In the wild-type AAV, these three proteins are translated from the p40 promoter to form a single mRNA. After this mRNA is synthesized, either a long or a short intron can be excised, resulting in the formation of a 2.3 kb or a 2.6 kb mRNA. The AAV capsid is composed of 60 capsid protein subunits (VP1, VP2, and VP3) that are arranged in an icosahedral symmetry in a ratio of 1:1:10, with an estimated size of 3.9 MDa.

As used herein, the term “cap gene” includes wild-type cap genes and derivatives thereof, and artificial cap genes which have equivalent functions.

The AAV cap gene sequences and Cap polypeptide sequences for AAV serotypes 1-9 are given herein in SEQ ID NOs: 1-18, respectively. As used herein, the term “cap gene” or Cap polypeptide-encoding sequence preferably includes, but is not limited to:
(a) a polynucleotide molecule whose nucleotide sequence comprises or consists of the nucleotide sequence given in any one of SEQ ID NOs: 1, 3, 5, 7, 9, 11, 13, 15 or 17 (preferably SEQ ID NO: 17);
(b) a polynucleotide molecule whose nucleotide sequence comprises or consists of a variant of the nucleotide sequence given in any one of SEQ ID NOs: 1, 3, 5, 7, 9, 11, 13, 15 or 17 (preferably SEQ ID NO: 17), the variant having at least 80%, 85%, 90%, 95% or 99% (preferably at least 95%) sequence identity to any one of SEQ ID NOs: 1, 3, 5, 7, 9, 11, 13, 15 or 17 (preferably SEQ ID NO: 17); and
(c) a polynucleotide molecule whose nucleotide sequence comprises or consists of a nucleotide sequence which encodes:
   (i) a polypeptide whose amino acid sequence is given in any one of SEQ ID NOs: 2, 4, 6, 8, 10, 12, 14, 16, or 18 (preferably SEQ ID NO: 18), or
   (ii) a variant of (i), the variant having at least 80%, 85%, 90%, 95% or 99% (preferably at least 95%) sequence identity to any one of SEQ ID NOs: 2, 4, 6, 8, 10, 12, 14, 16, or 18 (preferably SEQ ID NO: 18).

Preferably, the variant is or encodes one or more VP1, VP2 or VP3 polypeptides.

As used herein, the term “Cap mRNA” refers preferably to a mRNA having the nucleotide sequence of a cap gene, wherein T is replaced by U in the mRNA sequence. The antisense RNA will have a nucleotide sequence which is complementary (or substantially complementary) to that of the Cap mRNA.

AAV Rep mRNA is produced from an AAV rep gene. As used herein, the term “rep gene” refers to a gene that encodes one or more open reading frames (ORFs), wherein each of said ORFs encodes an AAV Rep non-structural protein, or variant or derivative thereof. These AAV Rep non-structural proteins (or variants or derivatives thereof) are involved in AAV genome replication and/or AAV genome packaging.

The wild-type rep gene comprises three promoters: p5, p19 and p40. Two overlapping messenger ribonucleic acids (mRNAs) of different lengths can be produced from p5 and from p19. Each of these mRNAs contains an intron which can be either spliced out or not using a single splice donor site and two different splice acceptor sites. Thus, six different mRNAs can be formed, of which only four are functional. The two mRNAs that fail to remove the intron (one transcribed from p5 and one from p19) read through to a shared terminator sequence and encode Rep78 and Rep52, respectively. Removal of the intron and use of the 5′-most splice acceptor site does not result in production of any functional Rep protein—it cannot produce the correct Rep68 or Rep40 proteins as the frame of the remainder of the sequence is shifted, and it will also not produce the correct C-terminus of Rep78 or Rep52 because their terminator is spliced out. Conversely, removal of the intron and use of the 3′ splice acceptor will include the correct C-terminus for Rep68 and Rep40, whilst splicing out the terminator of Rep78 and Rep52. Hence the only functional splicing either avoids splicing out the intron altogether (producing Rep78 and Rep52) or uses the 3′ splice acceptor (to produce Rep68 and Rep40). Consequently, four different functional Rep proteins with overlapping sequences can be synthesized from these promoters.

In the wild-type rep gene, the p40 promoter is located at the 3′ end. Transcription of the Cap proteins (VP1, VP2 and VP3) is initiated from this promoter in the wild-type AAV genome.

The four wild-type Rep proteins are Rep78, Rep68, Rep52 and Rep40. Hence the wild-type rep gene is one which encodes the four Rep proteins Rep78, Rep68, Rep52 and Rep40.

As used herein, the term “rep gene” includes wild-type rep genes and derivatives thereof; and artificial rep genes which have equivalent functions. The wild-type rep gene encodes Rep78, Rep68, Rep52 and Rep40 polypeptides.

The full wild-type AAV (serotype 2) rep gene nucleotide sequence is given in SEQ ID NO: 19. The wild-type AAV (serotype 2) Rep78, Rep68, Rep52 and Rep40 nucleotide sequences are given herein in SEQ ID NOs: 20, 22, 24 and 26, respectively. The wild-type AAV (serotype 2) Rep78, Rep68, Rep52 and Rep40 amino sequences are given herein in SEQ ID NOs: 21, 23, 25 and 27, respectively.

As used herein, the term “rep gene” or Rep polypeptide-encoding sequence preferably includes, but is not limited to:
(a) a polynucleotide molecule whose nucleotide sequence comprises or consists of the nucleotide sequence given in any one of SEQ ID NOs: 19, 20, 22, 24 or 26 (preferably, SEQ ID NO: 19); and
(b) a polynucleotide molecule whose nucleotide sequence comprises or consists of a variant of the nucleotide sequence given in any one of SEQ ID NOs: 19, 20, 22, 24 or 26 (preferably SEQ ID NO: 19), the variant having at least 80%, 85%, 90%, 95% or 99% (preferably at least 95%) sequence identity to any one of SEQ ID NOs: 19, 20, 22, 24, or 26 (preferably SEQ ID NO: 19); and
(c) a polynucleotide molecule whose nucleotide sequence comprises or consists of a nucleotide sequence which encodes:
   (i) a polypeptide whose amino acid sequence is given in any one of SEQ ID NOs: 21, 23, 25 or 27, (preferably SEQ ID NO: 21), or
   (ii) a variant of (i), the variant having at least 80%, 85%, 90%, 95% or 99% (preferably at least 95%) sequence identity to any one of SEQ ID NOs: 21, 23, 25 or 27 (preferably SEQ ID NO: 21).

Preferably, the variant is or encodes one or more Rep78, Rep68, Rep52 or Rep40 polypeptides.

As used herein, the term “Rep mRNA” refers preferably to a mRNA having the nucleotide sequence of a rep gene, wherein T is replaced by U in the mRNA sequence. The antisense RNA will have a nucleotide sequence which is complementary (or substantially complementary) to that of the Rep mRNA.

The rep and cap genes (and each of the protein-encoding ORFs therein) may be from one or more different viruses. For example, the rep gene may be from AAV2, whilst a cap gene may be from AAV5.

It is recognised by those in the art that the rep and cap genes of AAV vary by clade and isolate. The sequences of these genes from all such ciades and isolates are encompassed herein, as well as derivatives thereof.

The cell of the invention comprises:
(a) a first DNA molecule which comprises
   (i) a first promoter, operably-associated with
   (ii) a DNA molecule which encodes an antisense RNA which is capable of binding to an AAV cap mRNA,
and/or
(b) a second DNA molecule which comprises
   (i) a second promoter, operably-associated with
   (ii) a DNA molecule which encodes an antisense RNA which is capable of binding to an AAV rep mRNA,
and wherein the first and/or second DNA molecules are stably integrated into the cell's genome.

The first and second DNA molecules may be separate or contiguous. Preferably, the term “genome” relates to the cell's nuclear genome.

As used herein, the term “stably integrated” means that the first and second DNA molecules are integrated into the cell genome in a way such that the first and second DNA molecules will be passed from a parent cell to all daughter cells.

The first DNA molecule comprises:
(i) a first promoter, operably-associated with
(ii) a DNA molecule which encodes an antisense RNA which is capable of binding to an AAV cap mRNA.

An antisense RNA is produced from the first DNA molecule. The first promoter promotes the expression of the antisense RNA.

Preferably, the antisense RNA is capable of binding to and inhibiting translation of an AAV Cap mRNA (compared to the translation of a control AAV Cap mRNA in the absence of the antisense RNA).

In some embodiments, the antisense RNA is capable of binding to and increasing the degradation rate of an AAV Cap mRNA (compared to the degradation rate of a control AAV Cap mRNA in the absence of the antisense RNA).

The antisense RNA is a single-stranded RNA molecule. Preferably, the antisense RNA is a short hairpin RNA that is processed to produce an siRNA or a mature microRNA (miRNA).

miRNAs are short regulatory RNAs that function by guiding the miRNA-induced silencing complex (miRISC) to RNA targets bearing a complementary “seed” sequence.

The binding of a miRNA to the seed sequence results in attenuation of gene expression (protein production for coding genes); possible mechanisms include translational inhibition and target mRNA destabilization and decay.

The AAV Cap mRNA may be one or more of a VP1-encoding mRNA, a VP2-encoding mRNA and a VP3-encoding mRNA.

In some embodiments, the antisense RNA binds to an AAV Cap mRNA which encodes VP1. In some embodiments, the antisense RNA binds to an AAV Cap mRNA which encodes VP2. In some embodiments, the antisense RNA binds to an AAV Cap mRNA which encodes VP3.

In some embodiments, the antisense RNA binds to a region of an AAV Cap mRNA which is common to all mRNA which encode VP1, VP2 or VP3 (of a particular strain of AAV).

In other embodiments, the antisense RNA binds to the 5′ or 3′ UTR of an AAV Cap mRNA, preferably to the 3′ UTR. An untranslated region (or UTR) refers to either of two sections, one on each side of a coding sequence on a strand of mRNA. If it is found on the 5′ side, it is called the 5′ UTR (or leader sequence), or if it is found on the 3′ side, it is called the 3′ UTR (or trailer sequence). The 3′ UTR is found following the translation stop codon. The 3′ UTR plays a role in translation termination as well as post-transcriptional modification.

The degree of complementary nucleotide sequence identity between the antisense RNA (i.e. the anti-Cap mRNA antisense RNA) and the corresponding region of Cap mRNA is preferably at least 70%, 80%, 90%, 95% or 100%, preferably 100%.

The length of antisense RNA which has complementary sequence identity to the Cap mRNA is preferably 18-27 nucleotides, more preferably 20-22 nucleotides, and most preferably 21 nucleotides.

Preferably, the antisense RNA inhibits the translation of the Cap mRNA by at least 70%, preferably at least 80% or at least 90%. The degree of inhibition of the Cap mRNA may be assayed by Western blot, using an anti-Cap antibody.

Preferably, the antisense RNA increases the degradation rate of the Cap mRNA and thereby reduces translation and/or expression of the Cap polypeptide. The degree of Cap mRNA degradation is preferably at least 70%. mRNA degradation may be assayed by RT-QPCR against the Cap mRNA or Northern blotting against the same target.

In some embodiments, it is preferable that the antisense RNA increases the accumulation of the Cap mRNA in processing bodies. This can be determined by staining for the presence of Cap mRNA in fixed cells by microscopy using a labelled RNA that can hybridise to the Cap mRNA sequences.

The second DNA molecule comprises:
(i) a second promoter, operably-associated with
(ii) a DNA molecule which encodes an antisense RNA which is capable of binding to an AAV Rep mRNA.

An antisense RNA is produced from the second DNA molecule. The second promoter promotes the expression of the antisense RNA.

Preferably, the antisense RNA is capable of binding to and inhibiting translation of an AAV Rep mRNA (compared to the translation of a control AAV Rep mRNA in the absence of the antisense RNA).

In some embodiments, the antisense RNA is capable of binding to and increasing the degradation rate of an AAV Rep mRNA (compared to the degradation rate of a control AAV Rep mRNA in the absence of the antisense RNA).

The antisense RNA is a single-stranded RNA molecule. Preferably, the antisense RNA is a short hairpin RNA that is processed to produce an siRNA or a mature microRNA.

The AAV Rep mRNA may be one or more of a Rep78-encoding mRNA, a Rep68-encoding mRNA, a Rep52-encoding mRNA and a Rep40-encoding mRNA.

In some embodiments, the antisense RNA binds to a Rep78-encoding mRNA. In some embodiments, the antisense RNA binds to a Rep68-encoding mRNA. In some embodiments, the antisense RNA binds to a Rep52-encoding mRNA. In some embodiments, the antisense RNA binds to a Rep40-encoding mRNA.

In some embodiments, the antisense RNA binds to a region of an AAV Rep mRNA which is common to all mRNA which encode Rep78, Rep68, Rep52 and Rep40 (of a particular strain of AAV).

Preferably, the antisense RNA binds to the coding sequence of a Rep78-encoding mRNA and/or the coding sequence of a Rep68-encoding mRNA. In other embodiments, the antisense RNA binds to the 5′ or 3′ UTR of an AAV Rep mRNA, preferably to the 3′ UTR.

The degree of complementary nucleotide sequence identity between the antisense RNA (i.e. the anti-Rep mRNA antisense RNA) and the corresponding region of the Rep mRNA is preferably at least 70%, 80%, 90%, 95% or 100%, more preferably 100%.

The length of antisense RNA which has complementary sequence identity to the Rep mRNA is preferably 18-27 nucleotides, more preferably 20-22 nucleotides, and most preferably 21 nucleotides.

Preferably, the antisense RNA inhibits the translation of the Rep mRNA by at least 70%, preferably at least 80% or at least 90%. The degree of inhibition of the Rep mRNA may be assayed by Western blot, using an anti-Rep antibody.

Preferably, the antisense RNA increases the degradation rate of the Rep mRNA and thereby reduces translation and/or expression of the Rep polypeptide. The degree of Rep mRNA degradation is preferably at least 70%. mRNA degradation may be assayed by RT-QPCR against the Rep mRNA or Northern blotting against the same target.

In some embodiments, it is preferable that the antisense RNA increases the accumulation of the Rep mRNA in processing bodies. This can be determined by staining for the presence of Rep mRNA in fixed cells by microscopy using a labelled RNA that can hybridise to the Rep mRNA sequences.

The antisense RNA which is produced in the cell may be in the form of a Dicer-substrate which is cleaved and processed into a 21-23 nucleotide siRNA molecule in the cell.

The promoters which are operably-associated with the first and second DNA molecules are preferably constitutive promoters. Examples of constitutive promoters include the CMV, SV40, PGK (human or mouse), HSV TK, SFFV, Ubiquitin, Elongation Factor Alpha, CHEF-1, FerH, Grp78, RSV, Adenovirus E1A, CAG or CMV-Beta-Globin promoter, or a promoter derived therefrom.

The first and second promoters may be of the same sequence or same type of promoter, or different sequence or type.

The first and/or second promoter may be an RNA Polymerase III promoter. Examples of Pol III promoters that can be used are U6 and H1. When a Pol III promoter is used an appropriate terminator will also be used downstream of the antisense RNA that can terminate transcription.

In some embodiments, a single promoter drives the expression of the antisense RNA which binds to the Cap mRNA and the antisense RNA which binds to the Rep mRNA.

The production of antisense RNA against the AAV Cap mRNA and the production of antisense RNA against the AAV Rep mRNA may be independently or jointly controlled.

The cells of the invention are particularly useful for the production of recombinant adenoviruses (AVs). In some embodiments, the cell is one which additionally comprises a recombinant adenovirus comprising a recombinant adenovirus genome, wherein the recombinant adenovirus genome comprises (a) an AAV cap gene; and/or (b) an AAV rep gene. The AAV cap and AAV rep genes are preferably as defined herein. Preferably, the AAV cap and rep genes are both stably integrated into the genome of a recombinant adenovirus, more preferably inserted in the regions of the adenovirus that are normally occupied by the E1 or E3 genes.

The rep genes and the cap genes in the recombinant adenovirus may each independently be operably-associated with a promoter, e.g. a constitutive promoter, an inducible promoter, a repressible promoter, a minimal promoter, or with no promoter.

As used herein, the term “operably-associated” in the context of a promoter and a gene means that the promoter and the gene in question are located within a distance from each other which is sufficiently close for the promoter to promote transcription of the gene or antisense RNA. In some embodiments, the promoter and the gene are juxtaposed or are contiguous. In this context, the promoter is capable of driving transcription of the operably-associated gene, nucleic acid or DNA molecule to produce the antisense RNA.

Preferably, the promoter which is operably-associated with the AAV cap gene is a constitutive or inducible promoter, more preferably a constitutive promoter.

In some embodiments, the AAV cap gene is operably-associated with no promoter or with a minimal promoter.

Preferably, the promoter which is operably-associated with the AAV cap gene is selected based on the toxicity of the cap gene to the adenovirus and the expression levels required. The inventors have found that some cap genes can be expressed under the CMV promoter and have little to no impact on AV replication, e.g. AAV9. Conversely, some cap genes can only be inserted into AVs if driven by a minimal CMV promoter that has comparatively low expression, e.g. AAV6. Therefore, the promoter driving the cap gene will be based on experimentally testing a low- and high-expressing promoter, where the high-expressing promoter is the CMV promoter and the low expression is the minimal promoter region from the same promoter.

Preferably, the promoter which is operably-associated with the AAV rep gene is a weak promoter and only produces low or basal levels of transcription. The inventors have found that any attempts to insert a rep gene into an AV where a medium—(e.g. PGK) or high—(e.g. CMV) expressing promoter is highly toxic.

In some embodiments, the Rep polypeptide is expressed at a low, baseline or minimal level. In some embodiments, the AAV rep gene is not operably-associated with any functional promoter. As used herein, the term “low, baseline or minimal level” refers to a level of expression of the Rep78 polypeptide which is less than 50%, 40%, 30%, 20% or 10% of the level of expression of a wild-type Rep 78 polypeptide which is operably-associated with a wild-type p5 promoter (in a wad-type AAV rep gene). In this way, sufficient Rep polypeptide is provided in order to enable the production of at least some AAV, but the level of Rep polypeptide expression is insufficient to completely inhibit adenovirus replication.

Examples of constitutive promoters include the CMV, SV40, PGK (human or mouse), HSV TK, SFFV, Ubiquitin, Elongation Factor Alpha, CHEF-1, FerH, Grp78, RSV, Adenovirus E1A, CAG or CMV-Beta-Globin promoter, or a promoter derived therefrom.

Preferably, the rep gene has no promoter or is a minimal promoter region from the list derived above.

In some embodiments, the promoter is inducible or repressible by the inclusion of an inducible or repressible regulatory (promoter) element. For example, the promoter may be one which is inducible with doxycycline, tetracycline, IPTG or lactose.

The rep genes and the cap genes in the recombinant adenovirus may each also independently be operably-associated with a terminator, e.g. an SV40 polyadenylation signal.

WO2019/020992 discloses that transcription of the Late adenoviral genes can be regulated (e.g. inhibited) by the insertion of a repressor element into the Major Late Promoter. By “switching off” expression of the adenoviral Late genes, the cell's protein-manufacturing capabilities can be diverted toward the production of a desired recombinant protein or recombinant AAV particles. The contents of WO2019/020992 are specifically incorporated herein in their entirety.

In some embodiments, therefore, the recombinant adenovirus (i.e. adenoviral vector) comprises a repressible Major Late Promoter (MLP) and a plurality of adenoviral late genes, wherein the MLP comprises one or more repressor elements which are capable of regulating or controlling transcription of the adenoviral late genes, and wherein one or more of the repressor elements are inserted downstream of the MLP TATA box.

In other embodiments, the recombinant adenovirus (i.e. adenoviral vector) comprises:
(a) a plurality of adenoviral early genes, and
(b) a plurality of adenoviral late genes under the control of a Major Late Promoter (MLP), and
(c) a transgene (e.g. comprising AAV rep and cap genes),
wherein the MLP comprises one or more repressor elements which are capable of regulating or controlling transcription of the adenoviral late genes, and wherein one or more of the repressor elements are inserted downstream of the MLP TATA box.

Preferred features of these aspects of the invention include the following:
wherein the one or more repressor elements are inserted between the MLP TATA box and the +1 position of transcription.
wherein the repressor element is one which is capable of being bound by a repressor protein.
wherein a gene encoding a repressor protein which is capable of binding to the repressor element is encoded within the adenoviral genome.
wherein the repressor protein is transcribed under the control of the MLP.
wherein the repressor protein is the tetracycline repressor, the lactose repressor or the ecdysone repressor, preferably the tetracycline repressor (TetR).
wherein the repressor element is a tetracycline repressor binding site comprising or consisting of the sequence set forth in SEQ ID NO: 28.
wherein the nucleotide sequence of the MLP comprises or consists of the sequence set forth in SEQ ID NO: 29 or 30.
wherein the presence of the repressor element does not affect production of the adenoviral E2B protein.
wherein the adenoviral vector encodes the adenovirus L4 100K protein and wherein the L4 100K protein is not under control of the MLP.
wherein a transgene (e.g. comprising AAV rep and cap genes) is inserted within one of the adenoviral early regions, preferably within the adenoviral E1 region (instead of in a Transfer Plasmid).
wherein the transgene (e.g. comprising AAV rep and cap genes) comprises a Tripartite Leader (TPL) in its 5′-UTR.
wherein the transgene encodes a therapeutic polypeptide.
wherein the transgene encodes a virus protein, preferably a protein that is capable of assembly in or outside of a cell to produce a virus-like particle, preferably wherein the transgene encodes Norovirus VP1 or Hepatitis B HBsAG.

Preferably, one or more of the repressor elements are inserted downstream of the MLP TATA box.

Preferably, the transgene comprises AAV rep and cap genes.

In other embodiments, the invention provides a process for producing recombinant AV particles, the process comprising the steps:
(a) introducing, into a plurality of cells of the invention, a recombinant adenovirus comprising a recombinant adenovirus genome, wherein the recombinant adenovirus genome comprises:
   (i) an AAV cap gene; and/or
   (ii) an AAV rep gene,
(b) culturing the cells in a culture medium under conditions such that antisense RNAs which are capable of binding to AAV cap mRNA and antisense RNAs which are capable of binding to AAV rep mRNA are produced;
and optionally,
(c) purifying and/or isolating recombinant AV particles from the cells or from the culture medium.

In yet a further embodiment, there is provided a process for producing a recombinant cell, the process comprising the steps:
(A) introducing:
(a) a first DNA molecule which comprises
   (i) a first promoter, operably-associated with
   (ii) a DNA molecule which encodes an antisense RNA which is capable of binding to an AAV Cap mRNA,
and/or
(b) a second DNA molecule which comprises
   (i) a second promoter, operably-associated with
   (ii) a DNA molecule which encodes an antisense RNA which is capable of binding to an AAV Rep mRNA,
into a cell; and
(B) culturing the cell under conditions such that the first and/or second DNA molecules stably integrate into the genome of the cell.

Preferred cells are disclosed herein. Suitable conditions are well known in the art. The first and second DNA molecules may be separate or contiguous. Preferably, the first and second DNA molecules are introduced into the cell (together) in a single plasmid or vector.

As used herein, the term “introducing” includes transformation, and any form of electroporation, conjugation, infection, transduction or transfection, inter alia. The term “introduced” is similarly interpreted, mutatis mutandis. For example, the first and/or second DNA molecules may be integrated into the cell genome by random integration, through the use of a transposon comprising the first and/or second DNA molecules or in a lentivirus.

Preferably, the nucleic acid molecule(s) encoding the one or more DNA molecules encoding antisense RNA become stably integrated into the host cell genome. This can be achieved via the transfection of said DNA molecules using any suitable delivery method or transfection reagent that will allow DNA delivery to cells. Such suitable methods include cationic lipids such as polyethyleneimine (PEI) or electroporation.

Preferably, the nucleic acid molecule(s) encoding the one or more DNA molecules encoding antisense RNA additionally comprise a selection gene. Suitable selection genes and approaches are described below.

DNA transposons are natural and easily-controllable DNA delivery vehicles that can be used as tools for versatile gene delivery and gene discovery applications ranging from transgenesis to functional genomics and gene therapy. Transposons are simply organized: they encode a transposase protein in their simple genome flanked by inverted terminal repeats (ITRs) that carry transposase binding sites necessary for transposition. Transposons move through a “cut-and-paste” mechanism that involves excision of the element from the DNA and subsequent integration of the element into a new sequence environment.

DNA transposons are classified into different families depending on their sequence, Inverted Terminal Repeats, and/or target site duplications. The families in Subclass I are: Tc1/mariner, PIF/Harbinger, hAT, Mutator, Merlin, Transib, P, piggyBac and CACTA, and Sleeping Beauty. Helitron and Maverick transposons belong to Subclass II, since they are replicated and do not perform double-strand DNA breaks during their insertion. In some embodiments, the transposon is based upon a Class II, Subclass I transposon. In other embodiments, the transposon is based upon a piggyBac, Sleeping Beauty or a Tol2 transposon, or a variant or derivative thereof.

Retroviruses (including lentiviruses) are positive-sense RNA viruses that undergo a complex life cycle involving the reverse transcription of their genome into deoxyribonucleic acid (DNA), which subsequently becomes integrated into the host cell genome following viral infection. They are capable of inserting their genomes, as DNA, into almost any loci in the genome of target cells and mediating long-term expression of virus genes, with the DNA being copied into each daughter cell when the infected cell divides.

Although wild-type retro/lentiviruses can replicate in host cells, retro/lentivirus vectors are typically disabled in a range of ways to remove their ability to replicate and cause disease. The production and use of lentiviral vectors is described in WO2019/058108.

In some embodiments, the first and/or second DNA molecules are present in an adenoviral vector as disclosed in WO2019/020992 and/or as disclosed herein.

The production of stable cell lines in mammalian culture typically requires a method of selection to promote the growth of cells containing any exogenously-added DNA. Preferably, the first and/or second DNA molecules additionally comprise a selection gene or an antibiotic resistance gene. To this end, a range of genes are known that provide resistance to specific compounds when the DNA encoding them is inserted into a mammalian cell genome. Preferably, the selection gene is puromycin N-acetyl-transferase (Puro), hygromycin phosphotransferase (Hygro), blasticidin s deaminase (Blast) or Neomycin phosphotransferase (Neo). Each of these genes provides resistance to a small molecule known to be toxic to mammalian cells.

In a preferred embodiment of the invention, the resistance gene is Puro. This gene is particularly effective because many of the cell lines used in common tissue culture are not resistant; this cannot be said for Neo where many, particularly HEK 293 derivatives, are already Neo resistant due to previous genetic manipulations by researchers (e.g. HEK 293T cells). Puro selection also has the advantage of being toxic over a short time window (<72 hours), and hence it allows variables to be tested rapidly and cells that do not harbour the exogenous DNA to be inserted into the genome are rapidly removed from the culture systems. This cannot be said of some other selection methods such as Hygro, where toxicity is much slower onset.

The development of stable cell lines using selection genes (e.g. Puro) requires that the resistance gene must be expressed in the cells. This can be achieved through a variety of methods including, but not limited to, internal ribosome entry sites (IRES), 2A cleavage systems, alternative splicing, and dedicated promoters.

Preferably, the process steps of the inventions disclosed herein are carried out in the order specified.

There are many established algorithms available to align two amino acid or nucleic acid sequences. Typically, one sequence acts as a reference sequence, to which test sequences may be compared. The sequence comparison algorithm calculates the percentage sequence identity for the test sequence(s) relative to the reference sequence, based on the designated program parameters. Alignment of amino acid or nucleic acid sequences for comparison may be conducted, for example, by computer-implemented algorithms (e.g. GAP, BESTFIT, FASTA or TFASTA), or BLAST and BLAST 2.0 algorithms.

Percentage amino acid sequence identities and nucleotide sequence identities may be obtained using the BLAST methods of alignment (Altschul et al. (1997), “Gapped BLAST and PSI-BLAST: a new generation of protein database search programs”, Nucleic Acids Res. 25:3389-3402; and http://www.ncbi.nlm.nih.gov/BLAST). Preferably the standard or default alignment parameters are used.

Standard protein-protein BLAST (blastp) may be used for finding similar sequences in protein databases. Like other BLAST programs, blastp is designed to find local regions of similarity. When sequence similarity spans the whole sequence, blastp will also report a global alignment, which is the preferred result for protein identification purposes. Preferably the standard or default alignment parameters are used. In some instances, the “low complexity filter” may be taken off.

BLAST protein searches may also be performed with the BLASTX program, score=50, wordlength=3. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. (See Altschul et al. (1997) supra). When utilizing BLAST, Gapped BLAST, PSI-BLAST, the default parameters of the respective programs may be used.

With regard to nucleotide sequence comparisons, MEGABLAST, discontiguous-megablast, and blastn may be used to accomplish this goal. Preferably the standard or default alignment parameters are used. MEGABLAST is specifically designed to efficiently find long alignments between very similar sequences. Discontiguous MEGABLAST may be used to find nucleotide sequences which are similar, but not identical, to the nucleic acids of the invention.

The BLAST nucleotide algorithm finds similar sequences by breaking the query into short subsequences called words. The program identifies the exact matches to the query words first (word hits). The BLAST program then extends these word hits in multiple steps to generate the final gapped alignments. In some embodiments, the BLAST nucleotide searches can be performed with the BLASTN program, score=100, wordlength=12.

One of the important parameters governing the sensitivity of BLAST searches is the word size. The most important reason that blastn is more sensitive than MEGABLAST is that it uses a shorter default word size (11). Because of this, blastn is better than MEGABLAST at finding alignments to related nucleotide sequences from other organisms. The word size is adjustable in blastn and can be reduced from the default value to a minimum of 7 to increase search sensitivity.

A more sensitive search can be achieved by using the newly-introduced discontiguous megablast page (www.ncbi.nlm.nih.gov/Web/Newsltr/FallWinter02/blastlab.html). This page uses an algorithm which is similar to that reported by Ma et al. (Bioinformatics. 2002 March; 18(3): 440-5). Rather than requiring exact word matches as seeds for alignment extension, discontiguous megablast uses non-contiguous word within a longer window of template. In coding mode, the third base wobbling is taken into consideration by focusing on finding matches at the first and second codon positions while ignoring the mismatches in the third position. Searching in discontiguous MEGABLAST using the same word size is more sensitive and efficient than standard blastn using the same word size. Parameters unique for discontiguous megablast are: word size: 11 or 12; template: 16, 18, or 21; template type: coding (0), non-coding (1), or both (2).

In some embodiments, the BLASTP 2.5.0+ algorithm may be used (such as that available from the NCBI) using the default parameters.

In other embodiments, a BLAST Global Alignment program may be used (such as that available from the NCBI) using a Needleman-Wunsch alignment of two protein sequences with the gap costs: Existence 11 and Extension 1.

The nucleic acid molecules, plasmids and vectors of the invention may be made by any suitable technique. Recombinant methods for the production of the nucleic acid molecules and production cell lines of the invention are well known in the art (e.g. “Molecular Cloning: A Laboratory Manual” (Fourth Edition), Green, MR and Sambrook, J., (updated 2014)).

The disclosure of each reference set forth herein is specifically incorporated herein by reference in its entirety.

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: Infection of Cells Containing an siRNA Binding Rep or Cap mRNA

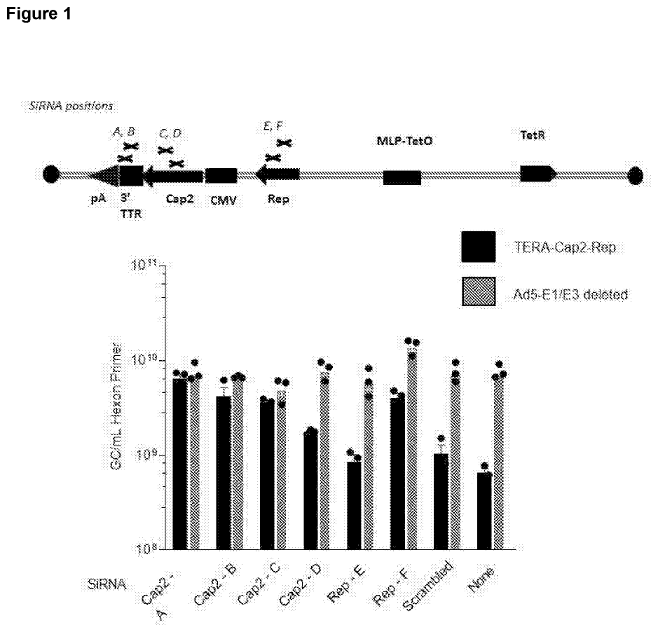

HEK 293 cells were seeded in Dulbecco's Modified Eagle (DMEM) media (10% FCS) into 48 well plates at 9×10⁴ cells per well. Cells were infected after 24 hours with an adenoviral vector containing AAV rep and cap2 or an adenoviral vector that did not contain AAV rep or cap genes. Wells containing the adenovirus containing the AAV Rep and Cap2 genes were then individually transfected with 5 picomoles per well of an siRNA binding either rep or cap complexed with 1.5 microlitres of lipofectamine RNAimax transfection reagent. These components were complexed according to the manufacturer's instructions. Wells containing the adenovirus that did not contain AAV genes were subjected to the same siRNAs to provide a baseline positive control. The binding sites of these siRNA are shown in FIG. 1. A scrambled siRNA was also used as a control as well as wells without any siRNA transfection.

The data shows that the adenovirus yields for both adenoviral vectors in the presence of each siRNA. These results show that the use of siRNA against both rep and cap can increase and improve the yield of an adenoviral vector containing AAV rep and cap genes. Interestingly, some siRNAs work more effectively than others, but this is expected and known to those in the art that are familiar with the use of siRNA molecules.

### Example 2: Protocol for Creating Stable Cell Lines Containing a DNA Encoding an Antisense RNA

In order to establish a stable cell line expressing DNA molecules encoding antisense RNA, HEK 293 cells can be seeded in Dulbecco's Modified Eagle (DMEM) media (10% FCS, 1% penicillin/streptomycin) into T25 flasks (10 cm or 6 cm dishes may also be used) 24 hr hours prior to transfection so as to be at 80% confluent at time of transfection.

Cells can then be transfected using the PEI method. Briefly, the transfection mixture can consist of 15 μg plasmid DNA in a 1:3 ratio with Branched PEI (25 KDa) respectively, which can then be added to two vials of 150 ul of DMEM (2% fetal calf serum (FCS)) Optimem media. Other media may be used, and preferably the media used would be Optimem to complex the DNA and would be free of both FCS and Penicillin and/or Streptomycin. The media/DNA mix and the media/PEI mix can then be combined and incubated for 20 minutes at room temperature to allow complex formation. At the time of transfection, the pre-existing media in which the cells have been seeded can be removed by aspiration and changed for fresh DMEM media containing 10% FCS. Transfection mixtures can then be added drop-wise into the flasks and gently swirled to evenly distribute the transfection complexes in the media.

24 hours post-transfection, the media and transfection mixtures in each flask can be removed by aspiration and replaced with DMEM (10% FCS, 1% penicillin/streptomycin) media containing puromycin which can then be added to the flasks at varying concentrations to determine the optimal antibiotic concentration required. For each DNA plasmid being tested, one flask can be maintained at each of the following concentrations: 0 μg/ml, 0.5 μg/ml, 1.5 μg/ml, 3 μg/ml, 5 μg/ml, 7.5 μg/ml and 10 μg/ml puromycin per ml of growth media.

Over the next 4 weeks, media in the flasks can be changed every 3-4 days, maintaining the same concentrations of puromycin relevant to each flask, and the flasks can be continuously evaluated for cell death and formation of cell foci/colonies. Formation of foci from single surviving cells can typically be observed in the flasks maintained at either 3 μg/ml and 5 μg/ml puromycin in flasks transfected with plasmids containing the DNA molecules encoding the antisense RNA and selection gene. After 4 weeks, two alternative approaches can be taken. The contents of each of flask can either be:
1. Gently trypsinized and passaged into larger T75 flasks, to make mixed population cell lines, maintaining the same puromycin concentration for each line. This can then be followed by single cell isolation by limited dilution or FACS to identify single monogenic cell lines; or
2. Single colonies can be encircled by a polycarbonate ring fixed temporarily to the flasks surface with sterile grease, followed by trypsinisation and colony isolation from within the ring and transfer to a 6-well plate containing the same media containing puromycin.
FACS is the more preferable method of clonal cell line isolation.

After cells derived from colonies reach sufficient confluence, they can be passaged with a 5-fold dilution; the remaining cells can be tested for the integration of the DNA by QPCR and expression of the siRNA by either a functional assay (e.g. transfection with a plasmid that has a reporter gene that has a target binding site for the antisense RNA and then measurement of report activity) or QPCR directly to measure the antisense RNA. The cell lines can be maintained from this point onwards at the same concentration of puromycin as originally selected in, or the puromycin can be withdrawn. Cell banks can be created and stored at −170° C. using the cells remaining from each passage. In some instances, it may be possible to increase the expression of the antisense RNA by increasing the puromycin concentration in 1-2 μg/ml increments, selecting for only the cells expressing the highest quantity of antisense RNA and puromycin resistance.

Cell lines may be created in the same way as described above but also using transposons. In this method, the approach is the same as described above, except that the DNA encoding the antisense RNA is flanked by the transposon ITRs and a plasmid encoding a transposase that binds the ITRs is simultaneously introduced into the cell line.

Cell lines may also be created in the same way as described above but using a lentiviral vector to introduce the DNA encoding the antisense RNA. Using a lentiviral vector containing the DNA encoding the antisense RNA means that the cells are infected with the lentiviral vector rather than transfection. After 24 hours post infection, cells can be selected in the same way as described above provided that the lentiviral vector contains a selection gene such as puromycin resistance.

### Example 3: Infection of a Cell Line Containing an Antisense RNA Against Rep and Cap

Cells containing a stably-integrated antisense RNA that binds to a rep or cap mRNA can be achieved by the introduction of a rep or cap gene into cells via suitable delivery method. This could be by transfection, electroporation or infection. In the instance where the cap and or rep genes encoding cap and rep mRNAs are inside an adenoviral vector, HEK293 cells stably containing the DNA encoding the antisense RNA can be seeded in suspension at 1.5×10⁶ cells/mL in a shake flask of appropriate size e.g. a 250 mL unbaffled polypropylene shakeflask, using and appropriate media e.g. BalanCD or CD293 media. Cells can then be infected with an adenoviral vector containing a rep or cap gene encoding a rep or cap mRNA. The production of the adenovirus can then be monitored by testing the cell supernatant or cell lysate by either qPCR.

### Example 4: Quantification of Total Adenoviral Genomes

For quantification of total adenovirus genomes in HEK293 cells, total DNA was extracted from culture media and cellular lysates using Purelink genomic DNA miniprep kit (Invitrogen, CA, USA). Five microlitres of DNA eluent were used in qPCR reactions using TaqMan Fast Advanced Master Mix (Applied Biosystems, CA, USA) in a StepOnePlus Real-Time PCR System (Applied Biosystems, CA, USA).

### Example 5: Quantification of Encapsulated Adenoviral Genomes

For quantification of genome encapsulated adenovirus particles, 2 μL of viral samples, harvested from culture medium or cell lysates, were treated with 1U of TURBO DNase (ThermoFisher Scientific, MA, USA) in a 20 μL reaction for 2-hours at 37° C. TURBO DNase was heat-inactivated at 75° C. for 10-minutes. Five microlitres of samples diluted at 1:200 using nuclease-free water were used in the PCR reaction to quantify encapsulated Ad5 using Ad5 hexon primers and probe. Standard curves for qPCR analyses were generated using a gBLOCK gene fragment suspended in nuclease-free water (Integrated DNA Technologies, IA, USA) and CT values of PCR reaction used to calculate DNA copy number by extrapolation to the standard curves (a qPCR standard of 3×10⁸-3×10¹ copies/well)

### Example 6: Quantitation of siRNA Expression Levels

siRNA expression levels in a stable cell line can be assessed by reverse transcription PCR (RT-PCR) using primers specific to the mature siRNA or microRNA or by using a labelled probe specific to the target RNA by northern blot. The level of the immature transcript can be measured using the same approaches but by designing primers and probes against a region of the expressed RNA that is not present in the mature siRNA or microRNA.

Dicer-substrate RNAs: chemically-synthesized 27mer duplex RNAs that have increased potency in RNA interference compared to traditional 21mer siRNAs. These will be cleaved and processed into 21-23 siRNA molecules in the cell.

| | | | SEQ |
|---|---|---|---|
| | | | ID |
| Samples | Bases | Sequence | NO: |
| SiRNA A | 25 | GCG UUU GUC UAU AUG UUA UUU | 31 |
| | | UCC A | |
| | 27 | UGG AAA AUA ACA UAU AGA CAA | 32 |
| | | ACG CAC | |
| SiRNA B | 25 | UGC GUU UGU CUA UAU GUU AUU | 33 |
| | | UUC C | |
| | 27 | GGA AAA UAA CAU AUA GAC AAA | 34 |
| | | CGC ACA | |
| SiRNA C | 25 | GUG GAC AUU GAA AAG GUC AUG | 35 |
| | | AUU A | |
| | 27 | UAA UCA UGA CCU UUU CAA UGU | 36 |
| | | CCA CAU | |
| SiRNA D | 25 | GUA CCU GUA UUA CUU GAG CAG | 37 |
| | | AAC A | |
| | 27 | UGU UCU GCU CAA GUA AUA CAG | 38 |
| | | GUA CUG | |
| SiRNA E | 25 | GAC AUG GAU CUG AAU CUG AUU | 39 |
| | | GAG C | |
| | 27 | GCU CAA UCA GAU UCA GAU CCA | 40 |
| | | UGU CAG | |
| SiRNA F | 25 | AAA GAG AAU CAG AAU CCC AAU | 41 |
| | | UCU G | |
| | 27 | CAG AAU UGG GAU UCU GAU UCU | 42 |
| | | CUU UGU | |

### Sequence Listing Free Text

<210> 1 <223> AAV1—Capsid Nucleotide Sequence (AF063497.1)

<210> 2 <223> AAV1—Capsid Protein Sequence

<210> 9 <223> AAV5—Capsid Nucleotide Sequence (AF085716.1)

<210> 10 <223> AAV5—Capsid Protein Sequence

<210> 11 <223> AAV6—Capsid Nucleotide Sequence (AF028704.1)

<210> 12 <223> AAV6—Capsid Protein Sequence

<210> 13 <223> AAV7—Capsid Nucleotide Sequence (AF513851.1)

<210> 14 <223> AAV7—Capsid Protein Sequence

<210> 15 <223> AAV8—Capsid Nucleotide Sequence (AF513852.1)

<210> 16 <223> AAV8—Capsid Protein Sequence

<210> 17 <223> AAV9—Capsid Nucleotide Sequence (AY530556.1)

<210> 18 <223> AAV9—Capsid Protein Sequence

<210> 28 <223> TetR binding site

<210> 29 <223> Modified MLP

<210> 30 <223> Modified MLP

<210> 31 <223> siRNA

<210> 32 <223> siRNA

<210> 33 <223> siRNA

<210> 34 <223> siRNA

<210> 35 <223> siRNA

<210> 36 <223> siRNA

<210> 37 <223> siRNA

<210> 38 <223> siRNA

<210> 39 <223> siRNA

<210> 40 <223> siRNA

<210> 41 <223> siRNA

<210> 42 <223> siRNA

1. A process for producing recombinant adenoviral (AV) particles, the process comprising the steps:
(A) introducing, into a plurality of cells a recombinant adenovirus comprising a recombinant adenovirus genome, wherein the recombinant adenovirus genome comprises:
(i) an AAV cap gene; and/or
(ii) an AAV rep gene,
and wherein the cells each comprise:
(a) a first DNA molecule which comprises:
(i) a first promoter, operably-associated with
(ii) a DNA molecule which encodes an antisense RNA which is capable of binding to an AAV Cap mRNA,
and/or
(b) a second DNA molecule which comprises:
(i) a second promoter, operably-associated with
(ii) a DNA molecule which encodes an antisense RNA which is capable of binding to an AAV Rep mRNA,
and wherein the first and/or second DNA molecules are stably integrated into the cell's genome;
(B) culturing the cells in a culture medium under conditions such that antisense RNAs which are capable of binding to AAV Cap mRNA and antisense RNAs which are capable of binding to AAV Rep mRNA are produced.

2. The process as claimed in claim 1, wherein the cell is from an adenovirus production cell line or an adenovirus manufacturing cell line.

3. The process as claimed in claim 1, wherein the cell is selected from the group consisting of HEK-293, HEK 293T, HEK-293E, HEK-293 FT, HEK-293S, HEK-293SG, HEK-293 FTM, HEK-293SGGD, HEK-293A MDCK, C127, A549, HeLa, CHO, mouse myeloma, PerC6, 911 and Vero cell lines, or a derivative thereof.

4. The process as claimed in claim 1, wherein the antisense RNA
which is capable of binding to an AAV Cap mRNA is also:
(i) capable of binding to and inhibiting translation of an AAV Cap mRNA; and/or
(ii) capable of binding to and increasing the degradation rate of an AAV Cap mRNA.

5. The process as claimed in claim 1, wherein the antisense RNA binds to the 5′ or 3′ UTR of a AAV Cap mRNA.

6. The process as claimed in claim 1, wherein the AAV Cap mRNA is:
(i) a VP1-encoding mRNA,
(ii) a VP2-encoding mRNA, or
(iii) a VP3-encoding mRNA.

7. The process as claimed in claim 1, wherein the antisense RNA
which is capable of binding to an AAV Rep mRNA is also:
(i) capable of binding to and inhibiting translation of an AAV Rep mRNA; and/or
(ii) capable of binding to and increasing the degradation rate of an AAV Rep mRNA.

8. The process as claimed in claim 1, wherein the AAV Rep mRNA is:
(i) a Rep78-encoding mRNA,
(ii) a Rep68-encoding mRNA,
(iii) a Rep52-encoding mRNA, or
(iv) a Rep40-encoding mRNA.

9. The process as claimed in claim 1, wherein the antisense RNA binds to:
(i) the coding sequence of a Rep78-encoding mRNA, and/or
(ii) the coding sequence of a Rep68-encoding mRNA.

10. The process as claimed in claim 1, wherein the degree of complementary nucleotide sequence identity between:
(i) the antisense RNA in (a) and the corresponding region of the Cap mRNA, and/or
(ii) the antisense RNA in (b) and the corresponding region of the Rep mRNA, is at least 70%, 80%, 90%, 95% or 100%.

11. The process as claimed in claim 1, wherein:
(i) the length of antisense RNA in (a) which has complementary sequence identity to the Cap mRNA, and/or
(ii) the length of antisense RNA in (b) which has complementary sequence identity to the Rep mRNA,
is 18-27 nucleotides, or 20-22 nucleotides or 21 nucleotides.

12. The process as claimed in claim 1, wherein:
(i) the antisense RNA which is capable of binding to an AAV Cap mRNA, and/or
(ii) the antisense RNA which is capable of binding to an AAV rep mRNA, is a siRNA or a miRNA.

13. The process as claimed in claim 1, wherein the first promoter and/or the second promoter is a constitutive promoter.

14. The process as claimed in claim 1, wherein the AAV cap gene in the recombinant adenovirus genome is operably-associated with no promoter or with a minimal promoter.

15. The process as claimed in claim 1, wherein the AAV Rep polypeptide is expressed from the AAV rep gene in the recombinant adenovirus genome at a low, baseline or minimal level.

16. The process as claimed in claim 1, wherein the recombinant adenovirus comprises a repressible Major Late Promoter (MLP) and a plurality of adenoviral late genes, wherein the MLP comprises one or more repressor elements which are capable of regulating or controlling transcription of the adenoviral late genes, and wherein one or more of the repressor elements are inserted downstream of the MLP TATA box.

17. A cell which comprises:
(A) a recombinant adenovirus comprising a recombinant adenovirus genome, wherein the recombinant adenovirus genome comprises:
(i) an AAV cap gene; and/or
(ii) an AAV rep gene,
(B) a first DNA molecule which comprises:
(i) a first promoter, operably-associated with
(ii) a DNA molecule which encodes an antisense RNA which is capable of binding to an AAV Cap mRNA,
and
(C) a second DNA molecule which comprises:
(i) a second promoter, operably-associated with
(ii) a DNA molecule which encodes an antisense RNA which is capable of binding to an AAV Rep mRNA,
and wherein the first and/or second DNA molecules are stably integrated into the cell's genome.

18. (canceled)

19. A process for producing a recombinant cell, the process comprising the steps:
(A) introducing
(a) a first DNA molecule which comprises
(i) a first promoter, operably-associated with
(ii) a DNA molecule which encodes an antisense RNA which is capable of binding to an AAV Cap mRNA,
and
(b) a second DNA molecule which comprises
(i) a second promoter, operably-associated with
(ii) a DNA molecule which encodes an antisense RNA which is capable of binding to an AAV Rep mRNA,
and
(c) a recombinant adenovirus comprising a recombinant adenovirus genome, wherein the recombinant adenovirus genome comprises:
(i) an AAV cap gene; and/or
(ii) an AAV rep gene,
into a cell;
and
(B) culturing the cell under conditions such that the first and/or second DNA molecules stably integrate into the genome of the cell.

20. The process as claimed in claim 1, wherein the process further comprises the step of:
(C) purifying and/or isolating recombinant AV particles from the cells or from the culture medium.


SEQUENCE LISTING (42 sequences)

SEQ ID NO: 1 - DNA, 2211 bp, Artificial Sequence
  AAV1 - Capsid Nucleotide Sequence (AF063497.1)
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 2 - protein, 736 aa, Artificial Sequence
  AAV1 - Capsid Protein Sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 3 - DNA, 2208 bp, adeno-associated virus 2
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 4 - protein, 735 aa, adeno-associated virus 2 [sequence omitted]

SEQ ID NO: 5 - DNA, 2211 bp, adeno-associated virus 3
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagggggctgtagatcagtctcctcaggaaccggactcatcatctggtgttggcaaatcgggcaaacagcctgccagaaaaagactaaatttcggtcagactggagactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttagaggggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactggaactgtcaatcatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtga

SEQ ID NO: 6 - protein, 736 aa, adeno-associated virus 3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 7 - DNA, 2205 bp, adeno-associated virus 4
atgactgacggttaccttccagattggctagaggacaacctctctgaaggcgttcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgcggagttccagcagcggcttcagggcgacacatcgtttgggggcaacctcggcagagcagtcttccaggccaaaaagagggttcttgaacctcttggtctggttgagcaagcgggtgagacggctcctggaaagaagagaccgttgattgaatccccccagcagcccgactcctccacgggtatcggcaaaaaaggcaagcagccggctaaaaagaagctcgttttcgaagacgaaactggagcaggcgacggaccccctgagggatcaacttccggagccatgtctgatgacagtgagatgcgtgcagcagctggcggagctgcagtcgagggcggacaaggtgccgatggagtgggtaatgcctcgggtgattggcattgcgattccacctggtctgagggccacgtcacgaccaccagcaccagaacctgggtcttgcccacctacaacaaccacctctacaagcgactcggagagagcctgcagtccaacacctacaacggattctccaccccctggggatactttgacttcaaccgcttccactgccacttctcaccacgtgactggcagcgactcatcaacaacaactggggcatgcgacccaaagccatgcgggtcaaaatcttcaacatccaggtcaaggaggtcacgacgtcgaacggcgagacaacggtggctaataaccttaccagcacggttcagatctttgcggactcgtcgtacgaactgccgtacgtgatggatgcgggtcaagagggcagcctgcctccttttcccaacgacgtctttatggtgccccagtacggctactgtggactggtgaccggcaacacttcgcagcaacagactgacagaaatgccttctactgcctggagtactttccttcgcagatgctgcggactggcaacaactttgaaattacgtacagttttgagaaggtgcctttccactcgatgtacgcgcacagccagagcctggaccggctgatgaaccctctcatcgaccagtacctgtggggactgcaatcgaccaccaccggaaccaccctgaatgccgggactgccaccaccaactttaccaagctgcggcctaccaacttttccaactttaaaaagaactggctgcccgggccttcaatcaagcagcagggcttctcaaagactgccaatcaaaactacaagatccctgccaccgggtcagacagtctcatcaaatacgagacgcacagcactctggacggaagatggagtgccctgacccccggacctccaatggccacggctggacctgcggacagcaagttcagcaacagccagctcatctttgcggggcctaaacagaacggcaacacggccaccgtacccgggactctgatcttcacctctgaggaggagctggcagccaccaacgccaccgatacggacatgtggggcaacctacctggcggtgaccagagcaacagcaacctgccgaccgtggacagactgacagccttgggagccgtgcctggaatggtctggcaaaacagagacatttactaccagggtcccatttgggccaagattcctcataccgatggacactttcacccctcaccgctgattggtgggtttgggctgaaacacccgcctcctcaaatttttatcaagaacaccccggtacctgcgaatcctgcaacgaccttcagctctactccggtaaactccttcattactcagtacagcactggccaggtgtcggtgcagattgactgggagatccagaaggagcggtccaaacgctggaaccccgaggtccagtttacctccaactacggacagcaaaactctctgttgtgggctcccgatgcggctgggaaatacactgagcctagggctatcggtacccgctacctcacccaccacctgtaa

SEQ ID NO: 8 - protein, 734 aa, adeno-associated virus 4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 9 - DNA, 2175 bp, Artificial Sequence
  AAV5 - Capsid Nucleotide Sequence (AF085716.1)
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 10 - protein, 724 aa, Artificial Sequence
  AAV5 - Capsid Protein Sequence
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 11 - DNA, 2211 bp, Artificial Sequence
  AAV6 - Capsid Nucleotide Sequence (AF028704.1)
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 12 - protein, 736 aa, Artificial Sequence
  AAV6 - Capsid Protein Sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 13 - DNA, 2214 bp, Artificial Sequence
  AAV7 - Capsid Nucleotide Sequence (AF513851.1)
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctgcaaagaagagaccggtagagccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagtgaaactgcaggtagtaccaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattcctcagtacggctacctgactctcaacaatggcagtcagtctgtgggacgttcctccttctactgcctggagtacttcccctctcagatgctgagaacgggcaacaactttgagttcagctacagcttcgaggacgtgcctttccacagcagctacgcacacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacctccaactttgaaaagcagactggtgtggactttgccgttgacagccagggtgtttactctgagcctcgccctattggcactcgttacctcacccgtaatctgtaa

SEQ ID NO: 14 - protein, 737 aa, Artificial Sequence
  AAV7 - Capsid Protein Sequence
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 15 - DNA, 2217 bp, Artificial Sequence
  AAV8 - Capsid Nucleotide Sequence (AF513852.1)
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 16 - protein, 738 aa, Artificial Sequence
  AAV8 - Capsid Protein Sequence
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 17 - DNA, 2214 bp, Artificial Sequence
  AAV9 - Capsid Nucleotide Sequence (AY530556.1)
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacggcaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtaattcctcgggaaattggcattgcgattccacatggctgggggacagagtcatcaccaccagcacccgaacctgggcattgcccacctacaacaaccacctctacaagcaaatctccaatggaacatcgggaggaagcaccaacgacaacacctactttggctacagcaccccctgggggtattttgacttcaacagattccactgccacttctcaccacgtgactggcagcgactcatcaacaacaactggggattccggccaaagagactcaacttcaagctgttcaacatccaggtcaaggaggttacgacgaacgaaggcaccaagaccatcgccaataaccttaccagcaccgtccaggtctttacggactcggagtaccagctaccgtacgtcctaggctctgcccaccaaggatgcctgccaccgtttcctgcagacgtcttcatggttcctcagtacggctacctgacgctcaacaatggaagtcaagcgttaggacgttcttctttctactgtctggaatacttcccttctcagatgctgagaaccggcaacaactttcagttcagctacactttcgaggacgtgcctttccacagcagctacgcacacagccagagtctagatcgactgatgaaccccctcatcgaccagtacctatactacctggtcagaacacagacaactggaactgggggaactcaaactttggcattcagccaagcaggccctagctcaatggccaatcaggctagaaactgggtacccgggccttgctaccgtcagcagcgcgtctccacaaccaccaaccaaaataacaacagcaactttgcgtggacgggagctgctaaattcaagctgaacgggagagactcgctaatgaatcctggcgtggctatggcatcgcacaaagacgacgaggaccgcttctttccatcaagtggcgttctcatatttggcaagcaaggagccgggaacgatggagtcgactacagccaggtgctgattacagatgaggaagaaattaaagccaccaaccctgtagccacagaggaatacggagcagtggccatcaacaaccaggccgctaacacgcaggcgcaaactggacttgtgcataaccagggagttattcctggtatggtctggcagaaccgggacgtgtacctgcagggccctatttgggctaaaatacctcacacagatggcaactttcacccgtctcctctgatgggtggatttggactgaaacacccacctccacagattctaattaaaaatacaccagtgccggcagatcctcctcttaccttcaatcaagccaagctgaactctttcatcacgcagtacagcacgggacaagtcagcgtggaaatcgagtgggagctgcagaaagaaaacagcaagcgctggaatccagagatccagtatacttcaaactactacaaatctacaaatgtggactttgctgtcaataccgaaggtgtttactctgagcctcgccccattggtactcgttacctcacccgtaatttgtaa

SEQ ID NO: 18 - protein, 737 aa, Artificial Sequence
  AAV9 - Capsid Protein Sequence
MAADGYLPDWLEDNLSEGIREWWALKPGAPKPKANQQKQDDGRGLVLPGYKYLGPFNGLDKGEPVNAADAAALEHGKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDEDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 19 - DNA, 1866 bp, adeno-associated virus 2
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaatag

SEQ ID NO: 20 - DNA, 1866 bp, adeno-associated virus 2
atgccggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtacctcagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaatag

SEQ ID NO: 21 - protein, 621 aa, adeno-associated virus 2
MPGFYEIVIKVPSDLDGHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 22 - DNA, 1589 bp, adeno-associated virus 2
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtacctcagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacagtag

SEQ ID NO: 23 - protein, 528 aa, adeno-associated virus 2
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYAD

SEQ ID NO: 24 - DNA, 1195 bp, adeno-associated virus 2
catggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaatag

SEQ ID NO: 25 - protein, 397 aa, adeno-associated virus 2
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 26 - DNA, 939 bp, adeno-associated virus 2
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacagattggctcgaggacactctctctag

SEQ ID NO: 27 - protein, 312 aa, adeno-associated virus 2
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 28 - DNA, 19 bp, Artificial Sequence
  TetR binding site
tccctatcagtgatagaga

SEQ ID NO: 29 - DNA, 107 bp, Artificial Sequence
  Modified MLP
cgccctcttcggcatcaaggaaggtgattggtttgtaggtgtaggccacgtgaccgggtgttcctgaaggggggctataaaaggtccctatcagtgatagagactca

SEQ ID NO: 30 - DNA, 107 bp, Artificial Sequence
  Modified MLP
cgccctcttcggcatcaaggaaggtgattggtttgtaggtgtaggccacgtgactccctatcagtgatagagaactataaaaggtccctatcagtgatagagactca

SEQ ID NO: 31 - RNA, 25 nt, Artificial Sequence
  siRNA
gcguuugucuauauguuauuuucca

SEQ ID NO: 32 - RNA, 27 nt, Artificial Sequence
  siRNA
uggaaaauaacauauagacaaacgcac

SEQ ID NO: 33 - RNA, 25 nt, Artificial Sequence
  siRNA
ugcguuugucuauauguuauuuucc

SEQ ID NO: 34 - RNA, 27 nt, Artificial Sequence
  siRNA
ggaaaauaacauauagacaaacgcaca

SEQ ID NO: 35 - RNA, 25 nt, Artificial Sequence
  siRNA
guggacauugaaaaggucaugauua

SEQ ID NO: 36 - RNA, 27 nt, Artificial Sequence
  siRNA
uaaucaugaccuuuucaauguccacau

SEQ ID NO: 37 - RNA, 25 nt, Artificial Sequence
  siRNA
guaccuguauuacuugagcagaaca

SEQ ID NO: 38 - RNA, 27 nt, Artificial Sequence
  siRNA
uguucugcucaaguaauacagguacug

SEQ ID NO: 39 - RNA, 25 nt, Artificial Sequence
  siRNA
gacauggaucugaaucugauugagc

SEQ ID NO: 40 - RNA, 27 nt, Artificial Sequence
  siRNA
gcucaaucagauucagauccaugucag

SEQ ID NO: 41 - RNA, 25 nt, Artificial Sequence
  siRNA
aaagagaaucagaaucccaauucug

SEQ ID NO: 42 - RNA, 27 nt, Artificial Sequence
  siRNA
cagaauugggauucugauucucuuugu